(19) United States
(12) Patent Application Publication
(10) Pub. No.: US 2025/0302311 A1
(43) Pub. Date: Oct. 2, 2025

(54) DEVICE FOR SENSING BIOMETRIC INFORMATION

(71) Applicant: SAMSUNG DISPLAY CO., LTD., YONGIN-SI (KR)

(72) Inventor: Chul KIM, Yongin-si (KR)

(21) Appl. No.: 18/925,392

(22) Filed: Oct. 24, 2024

(30) Foreign Application Priority Data
Mar. 26, 2024 (KR) 10-2024-0040919

(51) Int. Cl. A61B 5/00; A61B 5/0205

(52) U.S. Cl. CPC A61B 5/0082; A61B 5/0205; A61B 5/742

(57) ABSTRACT

An apparatus for detecting biometric information includes a sub-display panel configured to drive display pixels to emit light, and generate light sensing signals by sensing light using light sensing pixels, a biometric information detection circuit configured to measure and detect biometric information using the light sensing signals received from the sub-display panel, a main display panel configured to display at least a portion of the biometric information, and a display driving circuit configured to control a driving timing of the light sensing pixels and the display pixels of the sub-display panel.

### CROSS-REFERENCE TO RELATED APPLICATION

This application claims priority under 35 U.S.C. 119 from Korean Patent Application No. 10-2024-0040919 filed on Mar. 26, 2024, in the Korean Intellectual Property Office, the disclosure of which is herein incorporated by reference.

### BACKGROUND

### 1. Technical Field

The present disclosure relates to a device for sensing biometric information, and more particularly to a device capable of measuring biometric information using reflected light.

### 2. Discussion of Related Art

With the advancement of information-oriented societies, increasing demands are being placed on display devices in connection with the manner in which images may be displayed. A display device may include a liquid crystal display, a field emission display, or a light emitting display. In some cases, the display device may be implemented in a various electronic devices, including portable display devices such as smartphones. The electronic devices may have various functions such as image capturing, fingerprint recognition, or face recognition.

Recently, efforts have been made to combine a portable display device, such as a smart phone or a tablet PC with biometric detection devices. These biometric devices may measure biometric information such as a heart rate, heart rate variability, respiration, a cardiovascular disease, or oxygen saturation.

### SUMMARY

Aspects of the present disclosure provide a device capable of detecting a bio-signal of a user, such as a photoplethysmography signal, and measuring various pieces of biometric information such as a blood pressure, a heart rate, heart rate variability, a respiratory rate, or oxygen saturation.

In addition, aspects of the present disclosure provide a device capable of detecting the bio-signal by using a sub-display panel in which light emitting pixels and light sensing pixels are arranged, and displaying the biometric information using a main display panel.

However, aspects of the present disclosure are not restricted to those set forth herein. The above and other aspects of the present disclosure will become more apparent to one of ordinary skill in the art to which the present disclosure pertains by referencing the detailed description of the present disclosure given below.

According to an embodiment of the disclosure, an apparatus for detecting biometric information includes a sub-display panel configured to drive a plurality of display pixels to emit light, and generate light sensing signals by sensing light using a plurality of light sensing pixels, a biometric information detection circuit configured to measure and detect a user's biometric information using the light sensing signals received from the sub-display panel, a main display panel configured to display at least a portion of the biometric information, and a display driving circuit configured to control a driving timing of the plurality of light sensing pixels and the plurality of display pixels of the sub-display panel.

In an embodiment, the sub-display panel comprises a first bio-signal detection panel comprising a first emission area in which first display pixels of the plurality of display pixels are arranged and a first light sensing area in which first light sensing pixels of the plurality of light sensing pixels are arranged, and a circuit board coupled and electrically connected to a first end portion of the first bio-signal detection panel, wherein the biometric information detection circuit and the display driving circuit are mounted on the circuit board.

In an embodiment, the first display pixels are arranged in a matrix structure in the first emission area of the first bio-signal detection panel, the first light sensing area of the first bio-signal detection panel is formed at a center portion of the first emission area to have a perimeter smaller than that of the first emission area, and the first light sensing pixels are arranged in a matrix structure in the first light sensing area of the first bio-signal detection panel.

According to an embodiment of the disclosure, an apparatus for detecting biometric information includes a sub-display panel configured to drive display pixels to emit light, and detect light sensing signals by sensing light using light sensing pixels, the sub-display panel comprising a biometric information detection circuit configured to measure and detect biometric information of a user using the light sensing signals received through the sub-display panel, and a display driving circuit configured to control a driving timing of the light sensing pixels and the display pixels of the sub-display panel, and a main display panel configured to display the biometric information, wherein the main display panel is formed in a mobile display device in a state separated from the sub-display panel, and the sub-display panel further comprises at least one short-range wireless communication module configured to transmit the biometric information detected through the biometric information detection circuit to the mobile display device in which the main display panel is formed.

According to an embodiment of the disclosure, an apparatus for detecting biometric information includes a sub-display panel configured to drive a plurality of display pixels to emit light, and generate light sensing signals by sensing light reflected from a body part of a user using a plurality of light sensing pixels, a biometric information detection circuit configured to measure and detect biometric information of the user using the light sensing signals received from the sub-display panel, a main display panel configured to display at least a portion of the biometric information, and a display driving circuit configured to control a driving timing of the plurality of light sensing pixels and the plurality of display pixels of the sub-display panel, wherein the display driving circuit generates electrical signals comprising gate signals, a data voltage, and a driving voltage for driving the sub-display panel, and the biometric information detection circuit measures and detects the user's biometric information using light sensing signals received from the sub-display panel.

In accordance with a device according to embodiments, when light emitted from light emitting pixels of a sub-display panel is reflected from a user's body part, the reflected light may be detected by a light sensing pixel of a sub-display panel, and a bio-signal of the user, such as a pulse wave signal or the like may be detected. Further, a biometric information measurement result based on the bio-signal may be displayed on the main display panel.

Accordingly, the bio-signal of the user may be more accurately detected using the sub-display panel, and the user's various biometric information may be detected and confirmed by using various mobile display devices such as watch-type display devices, smartphones, laptops, and pads as the main display panel. In particular, by detecting the bio-signals by using the sub-display panel separate from the main display panel that displays images, the accuracy and reliability of biometric information detection may be further improved.

However, effects according to embodiments of the present disclosure are not limited to those exemplified above and various other effects are incorporated herein.

### BRIEF DESCRIPTION OF THE DRAWINGS

The above and other aspects and features of the present disclosure will become more apparent by describing in detail embodiments thereof with reference to the attached drawings, in which:

FIG. 1 is a plan view showing an apparatus according to an embodiment of the present disclosure;

FIG. 2 is a side view specifically showing the configuration of the apparatus shown in FIG. 1;

FIG. 3 is a layout diagram schematically showing an example of the main display panel in FIGS. 1 and 2;

FIG. 4 is a circuit diagram of an embodiment illustrating display pixels formed in the display areas of the main display panel and the sub-display panel;

FIG. 5 is a circuit diagram of an embodiment of a light sensing pixel formed in a light sensing area of a sub-display panel;

FIG. 6 is a diagram illustrating a biometric information detection process using the apparatus according to an embodiment;

FIG. 7 is a diagram illustrating a bio-signal detection process and biometric information confirmation process using the display device of FIG. 6;

FIG. 8 is a diagram illustrating a biometric information detection process using the apparatus according to an embodiment;

FIG. 9 is a graph illustrating a method for calculating blood pressure information among biometric information according to an embodiment;

FIG. 10 is a graph illustrating a method for calculating blood pressure information among biometric information according to an embodiment;

FIG. 11 is a graph illustrating a method for calculating information on a heart rate and respiration among biometric information according to an embodiment;

FIG. 12 is a graph illustrating a method for calculating information on blood vessel elasticity among biometric information according to an embodiment;

FIG. 13 is a graph illustrating a method for calculating information on a cardiovascular disease among biometric information according to an embodiment;

FIG. 14 is a graph illustrating a method for calculating information on a cardiovascular disease among biometric information according to an embodiment;

FIG. 15 is a graph illustrating a method for calculating information on oxygen saturation among biometric information according to an embodiment;

FIG. 16 is a diagram illustrating biometric information measurement results through the main display panel of the display device according to an embodiment;

FIG. 17 is a plan view showing an apparatus according to an embodiment of the present disclosure;

FIG. 18 and FIG. 19 are diagrams illustrating a biometric information detection process using the apparatus of FIG. 17;

FIG. 20 is a plan view showing an apparatus according to an embodiment of the present disclosure;

FIG. 21 is a diagram specifically illustrating a display pixel disposition structure in area A illustrated in FIG. 20;

FIG. 22 is a diagram specifically illustrating the disposition structure of display pixels according to stretch deformation of area A illustrated in FIG. 21; and

FIG. 23 is a plan view showing a display device according to an embodiment of the present disclosure.

### DETAILED DESCRIPTION

The present invention will now be described more fully hereinafter with reference to the accompanying drawings, in which preferred embodiments of the invention are shown. Aspects of the present invention may, however, be embodied in different forms and should not be construed as limited to embodiments set forth herein. Rather, embodiments are provided so that this disclosure will be thorough and complete, and will fully convey the scope of the invention to those skilled in the art.

It will also be understood that when a layer is referred to as being “on” another layer or substrate, the layer can be directly on the other layer or substrate, or intervening layers may also be present. In contrast, when an element is referred to as being “directly on” another element, there may be no intervening elements present. The same reference numbers indicate the same components throughout the specification.

It will be understood that, although the terms “first,” “second,” etc. may be used herein to describe various elements, these elements should not be limited by these terms. These terms are only used to distinguish elements. For instance, a first element discussed below could be termed a second element without departing from the teachings of the present invention. Similarly, the second element could also be termed the first element.

Features of different embodiments of the present disclosure may be implemented individually or combined with each other, in part or in whole. Embodiments may be implemented independently of each other or may be implemented together in an association.

Hereinafter, embodiments will be described in detail with reference to the accompanying drawings.

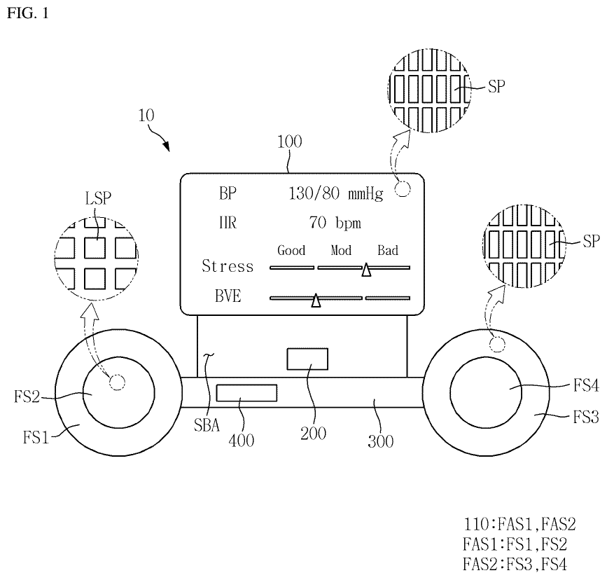

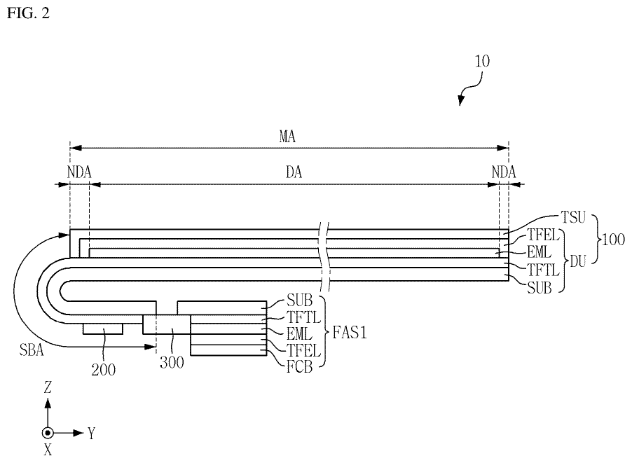

FIG. 1 is a plan view showing an apparatus according to an embodiment of the present disclosure. FIG. 2 is a side view specifically showing a configuration of the apparatus shown in FIG. 1.

Referring to FIG. 1 and FIG. 2, a display device 10 includes a main display panel 100, a sub-display panel 110, a display driving circuit 200, a circuit board 300, and a biometric information detection circuit 400.

The main display panel 100 and the sub-display panel 110 may include a plurality of display pixels SP. The sub-display panel 110 may drive the plurality of display pixels SP to emit light. The sub-display panel 110 may generate a light sensing signal by sensing light using light sensing pixels LSP. For example, the light may be reflected from a user.

The sub-display panel 110 may include a first bio-signal detection panel FAS1 and a second bio-signal detection panel FAS2. The first bio-signal detection panel FAS1 may include a first emission area FS1 in which the display pixels SP are arranged and a first light sensing area FS2 in which the light sensing pixels LSP are arranged. In addition, the second bio-signal detection panel FAS2 may include a second emission area FS3 in which the display pixels SP are arranged and a second light sensing area FS4 in which the light sensing pixels LSP are arranged.

Each of the first and second bio-signal detection panels FAS1 and FAS2 may be formed in a polygonal shape. For example, the first and second bio-signal detection panels FAS1 and FAS2 may be formed as a triangular shape, a quadrilateral shape, a pentagonal shape, or a hexagonal shape. In another example, the first and second bio-signal detection panels FAS1 and FAS2 may be formed having a rounded portion. For example, the first and second bio-signal detection panels FAS1 and FAS2 may be formed in a circular or elliptical planar shape. Hereinafter, an example in which the planar shape of each of the first and second bio-signal detection panels FAS1 and FAS2 is formed in a circular shape will be described. However, the planar shape of each of the first and second bio-signal detection panels FAS1 and FAS2 is not limited to embodiments illustrated in the drawings. For example, the planar shape of each of the first and second bio-signal detection panels FAS1 and FAS2 may be formed in different shapes. In another example, the planar shape of each of the first and second bio-signal detection panels FAS1 and FAS2 may not be formed in a circular shape, but may be formed in a square shape or a rectangular square having a short side in a first direction and a long side in a second direction orthogonal to the first direction. In this case, a corner at which a side in the first direction and a side in the second direction meet may be formed at a right angle or may be formed to be rounded to have a predetermined curvature.

The first and second bio-signal detection panels FAS1 and FAS2 may be a light emitting display panel, such as an organic light emitting display panel using an organic light emitting diode, a quantum dot light emitting display panel including a quantum dot light emitting layer, an inorganic light emitting display panel including an inorganic semiconductor, or an ultra-small light emitting display panel using an ultra-small light emitting diode (a micro or nano light emitting diode (micro LED or nano LED)). Hereinafter, an example with be described in which the first and second bio-signal detection panels FAS1 and FAS2 are organic light emitting display panels. That is, the organic light emitting display pixels SP may be formed in the first emission area FS1 and the second emission area FS3 of the first and second bio-signal detection panels FAS1 and FAS2. However, the first and second bio-signal detection panels FAS1 and FAS2 of the example are not limited to examples described herein.

The first and second bio-signal detection panels FAS1 and FAS2 may be formed flat, but are not limited thereto. For example, the first and second bio-signal detection panels FAS1 and FAS2 may include curved portions with a constant curvature or a changing curvature. In addition, the first and second bio-signal detection panels FAS1 and FAS2 may be flexible to be curved, bent, folded, stretched, or rolled.

The first emission area FS1 of the first bio-signal detection panel FAS1 may be formed in a planar shape such as a rectangular shape, a square shape, or a circular shape, and the display pixels SP may be arranged in a matrix structure in the first emission area FS1.

The first light sensing area FS2 of the first bio-signal detection panel FAS1 may have a perimeter smaller than a perimeter of the first emission area FS1 and may be formed at the center portion of the first emission area FS1, and the light sensing pixels LSP may be arranged in a matrix structure in the first light sensing area FS2.

An outer edge of the first emission area FS1 may have a same shape or a different shape from a shape of the first light sensing area FS2. For example, the outer edge of the first emission area FS1 and the first light sensing area FS2 may both have circular shapes. In another example, the outer edge the first emission area FS1 may have a circular shape and the first light sensing area FS2 may have a rectangular shape. In this case, an inner edge of the first emission area FS1 may have the same shape of the first light sensing area FS2, that is a rectangular shape.

The second emission area FS3 of the second bio-signal detection panel FAS2 may be formed in a planar shape such as a rectangular shape, a square shape, or a circular shape, and the display pixels SP may be arranged in a matrix structure in the second emission area FS3.

The second light sensing area FS4 of the second bio-signal detection panel FAS2 may have a perimeter smaller than a perimeter of the second emission area FS3 and may be formed at the center portion of the second emission area FS3, and the light sensing pixels LSP may be arranged in a matrix structure in the second light sensing area FS4.

An outer edge of the second emission area FS3 may have a same shape or a different shape from a shape of the second light sensing area FS4. For example, the outer edge of the second emission area FS3 and the second light sensing area FS4 may both have circular shapes. In another example, the outer edge the second emission area FS3 may have a circular shape and the second light sensing area FS4 may have a rectangular shape. In this case, an inner edge of the second emission area FS3 may have the same shape of the second light sensing area FS4, that is a rectangular shape.

The first bio-signal detection panel FAS1 and the second bio-signal detection panel FAS2 may be coupled or assembled to a first end portion and a second end portion of the circuit board 300, respectively, and may be electrically connected to the circuit board 300.

A sub-region SBA may be formed on a side surface or rear surface of the circuit board 300. For example, the sub-region SBA may be formed on a side surface or rear surface of the circuit board 300 by using a flexible film. The circuit board 300 may be formed of a flexible printed circuit board, a printed circuit board, or a chip on film.

The first and second bio-signal detection panels FAS1 and FAS2 may be electrically connected to the display driving circuit 200 and the biometric information detection circuit 400 through the circuit board 300. The first and second bio-signal detection panels FAS1 and FAS2 may receive one or more of gate signals, data voltages, or driving voltages through the circuit board 300.

The display driving circuit 200 may generate electrical signals to control a driving timing of the sub-display panel 110. The display driving circuit 200 may generate electrical signals for driving the first and second bio-signal detection panels FAS1 and FAS2. For example, the display driving circuit 200 may generate one or more electrical signals such as gate signals, data voltages, or driving voltages for driving the first and second bio-signal detection panels FAS1 and FAS2.

The biometric information detection circuit 400 and the display driving circuit 200 may be formed as an integrated circuit (IC). The biometric information detection circuit 400 may be disposed on the circuit board 300. The display driving circuit 200 may be disposed on the flexible film of the sub-region SBA by using a chip on glass (COG) method, a chip on plastic (COP) method, or an ultrasonic bonding method. However, the present disclosure is not limited thereto. For example, the biometric information detection circuit 400 and the display driving circuit 200 may be formed as an IC and disposed on the circuit board 300.

The main display panel 100 may be coupled to, or assembled with the flexible film of the sub-region SBA. Alternatively, the main display panel 100 may be separated from the first and second bio-signal detection panels FAS1 and FAS2 and the circuit board 300. In another alternative, the main display panel 100 may be mounted directly on the circuit board 300.

The main display panel 100 may be formed in a rectangular planar shape with perpendicular pairs of side. For example, the rectangular planar shape may have long first sides and short second sides, perpendicular to the long first sides. A corner at which two sides meet may be formed at a right angle or may be formed to be rounded at a predetermined curvature. The planar shape of the main display panel 100 is not limited to the rectangular shape, and may be formed in another polygonal shape, a circular shape or an elliptical shape. The main display panel 100 may be formed to be flat, but is not limited thereto. For example, the main display panel 100 may include a curved portion formed at left and right ends and having a constant curvature or a changing curvature. In addition, the main display panel 100 may be formed flexibly to be curved, bent, folded, or rolled.

Referring to FIG. 2, the substrate SUB of the main display panel 100 may include the main region MA and the sub-region SBA.

The main region MA may include a display area DA for displaying an image and a non-display area NDA that is a peripheral area of the display area DA.

The non-display area NDA may be disposed adjacent to the display area DA. The non-display area NDA may be an area outside the display area DA. The non-display area NDA may be disposed to surround the display area DA. The non-display area NDA may be an edge area of the main display panel 100.

The display area DA may occupy a majority of the main region MA. The display area DA may be disposed at a center portion of the main region MA. The display area DA may be disposed asymmetrically within the main region MA. For example, a width of the non-display area NDA may be different on different sides of the main region MA. In the display area DA, a plurality of display pixels SP may be arranged in a matrix structure, and an image may be displayed by the plurality of display pixels SP. In other words, an image may be displayed in the display area DA when light is emitted from the emission area or an opening area of each of the display pixels SP. To this end, the display pixels SP of the display area DA may include a pixel circuit including a switching element (e.g., a thin film transistor), a pixel defining film defining an emission area or an opening area, and a self-light emitting element.

The non-display area NDA may be disposed at an outer area of the display area DA or an area outside the display area DA. The non-display area NDA may be defined as an edge area of the main region MA of the main display panel 100. A gate driver (not illustrated) that supplies gate signals to the gate lines and fan-out lines (not illustrated) that connect the display driving circuit 200 to the display area DA may be formed in the non-display area NDA.

The sub-region SBA of the main display panel 100 may extend from the sub-region SBA of the first and second bio-signal detection panels FAS1 and FAS2. The sub-region SBA may protrude from a side of the main region MA. The sub-region SBA of the main display panel 100 may be coupled to, or assembled with, the circuit board 300 of the first and second bio-signal detection panels FAS1 and FAS2.

The substrate SUB of each of the first and second bio-signal detection panels FAS1 and FAS2 may be a base substrate or a base member. The substrate SUB may be a flexible substrate, which may be bent, folded or rolled. For example, the substrate SUB may include a glass material or a metal material, but is not limited thereto. For another example, the substrate SUB may include a polymer resin such as polyimide (PI).

A thin film transistor layer TFTL may be disposed on the substrate SUB of each of the first and second bio-signal detection panels FAS1 and FAS2, including the main display panel 100. The thin film transistor layer TFTL may include a plurality of thin film transistors forming a pixel circuit of the display pixels SP and the light sensing pixels LSP. The thin film transistor layer TFTL may further include gate lines, data lines, power lines, gate control lines, and fan-out lines that connect the display driving circuit 200 to the data lines, and lead lines that connect the display driving circuit 200 to a pad portion. Meanwhile, when a separate gate driving circuit is formed on the main display panel 100 or the first and second bio-signal detection panels FAS1 and FAS2, the gate driving circuit 210 may also include thin film transistors.

The light emitting element layer EML may be disposed on each thin film transistor layer TFTL. The light emitting element layer EML may include a plurality of light emitting elements and a pixel defining layer defining the display pixels SP. The plurality of light emitting elements may include a first electrode, a light emitting layer, and a second electrode that may be sequentially stacked and may emit light. The plurality of light emitting elements of the light emitting element layer EML may be disposed in the display area DA, the first emission area FS1 of the first bio-signal detection panel FAS1, and the second emission area FS3 of the second bio-signal detection panel FAS2.

The encapsulation layer TFEL may be disposed on a top surface and a side surface of each of the light emitting element layers EML. The encapsulation layer TFEL may protect each of the light emitting element layers EML. Each encapsulation layer TFEL may include at least one inorganic layer and at least one organic layer for encapsulating each light emitting element layer EML.

A touch sensing unit TSU may sense a touch. For example, the touch sensing unit TSU may sense a touch of a user's body part. The touch sensing unit TSU may be formed on the encapsulation layer TFEL of the main display panel 100. The touch sensing unit TSU may include a plurality of touch electrodes for sensing a user's touch in a capacitive manner, and touch lines connecting the plurality of touch electrodes to the biometric information detection circuit 400. For example, the touch sensing unit TSU may sense a user's touch by a self-capacitance method or a mutual capacitance method.

The display driving circuit 200 may generate electrical signals for driving the main display panel 100. The display driving circuit 200 may generate electrical signals such as control signals and data voltages for driving the main display panel 100. The display driving circuit 200 may transmit the electrical signals to the main display panel 100. Here, in addition to the display driving circuit 200 for driving the first and second bio-signal detection panels FAS1 and FAS2, the display driving circuit 200 for driving the main display panel 100 may be formed separately. However, for purposes of example, the driving of the main display panel 100 and the first and second bio-signal detection panels FAS1 and FAS2 is described using the display driving circuit 200.

The biometric information detection circuit 400, or another processor of the display device 10, may execute an application program. For example, the biometric information detection circuit 400, running the application program may perform a pulse wave signal detection process. For example, the biometric information detection circuit 400 may measure the user's biometric information by using light sensing signals received from the sub-display panel 110.

Specifically, the biometric information detection circuit 400 may receive light sensing signals through at least one bio-signal detection panel among the first and second bio-signal detection panels FAS1 and FAS2. Here, the biometric information detection circuit 400 may receive light sensing signals from the light sensing pixels LSP formed in the first light sensing area FS2 of the first bio-signal detection panel FAS1 and the second light sensing area FS4 of the second bio-signal detection panel FAS2.

The biometric information detection circuit 400 may detect photoplethysmography signals, among bio-signals corresponding to a magnitude change of the light sensing signals received from the light sensing pixels LSP. The photoplethysmography signals may be pulse wave signals.

In addition to pulse wave signals, bio-signals may further include electromyography signals or brain wave signals. However, the signals are not limited to examples described herein, and other bio-signals may be detected. Hereinafter, an example in which the biometric information detection circuit 400 detects and analyzes the pulse wave signals among the bio-signals and measures the user's biometric information will be described. The user's biometric information may include information such as a blood pressure, a heart rate, heart rate variability, a respiratory rate, blood vessel elasticity, occurrence or non-occurrence of a cardiovascular disease, or oxygen saturation.

The display driving circuit 200 may be used to drive the pulse wave signal detection process, which may be used by an application program such that the user's pulse wave signals may be accurately detected. Accordingly, the biometric information detection circuit 400 may accurately sample and select the pulse wave signals detected by analyzing the pulse wave signals. In particular, the biometric information detection circuit 400 may periodically analyze the pulse wave signals to measure biometric information. The biometric information may include a blood pressure, a heart rate, heart rate variability, a respiratory rate, blood vessel elasticity, occurrence or non-occurrence of a cardiovascular disease, or oxygen saturation.

The display driving circuit 200 may receive the biometric information through the biometric information detection circuit 400. Further, the display driving circuit 200 may display the measurement results of the biometric information using the main display panel 100.

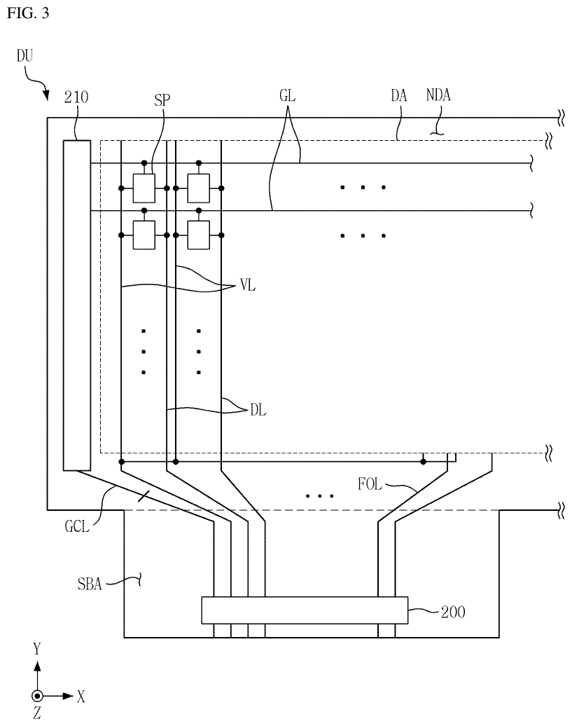

FIG. 3 is a layout diagram schematically showing an example of the main display panel in FIG. 1 and FIG. 2. Specifically, FIG. 3 is a layout diagram illustrating the display area DA and the non-display area NDA of the display unit DU, which may be formed before the touch sensing unit TSU is formed.

The display area DA, which is an area for displaying an image, may be defined as the central area of the main display panel 100. The display area DA may include a plurality of display pixels SP, a plurality of gate lines GL, a plurality of data lines DL, and a plurality of power lines VL. Each of the plurality of display pixels SP may be defined as a unit that may output light. A display pixel SP may be the smallest unit that may output light.

Referring to FIG. 3, the plurality of display pixels SP may be disposed in the first emission area FS1 of the first bio-signal detection panel FAS1 and the second emission area FS3 of the second bio-signal detection panel FAS2 in the same manner as the display area DA of the main display panel 100. That is, the first emission area FS1 of the first bio-signal detection panel FAS1 and the second emission area FS3 of the second bio-signal detection panel FAS2 may also include a plurality of display pixels SP, a plurality of gate lines GL, a plurality of data lines DL, and a plurality of power lines VL. Further, each of the plurality of display pixels SP may be defined as a unit that outputs light. Accordingly, the description of the layout structure of the first emission area FS1 of the first bio-signal detection panel FAS1 and the second emission area FS3 of the second bio-signal detection panel FAS2 will be substantially the same as a description of the display area DA and the non-display area NDA of FIG. 3. Repetitive descriptions may be omitted or substantially simplified.

The plurality of gate lines GL may supply scan signals or gate signals received from the gate driving circuit 210 to the plurality of display pixels SP. The plurality of gate lines GL may extend in an X-axis direction and may be spaced apart from each other in the Y-axis direction that crosses the X-axis direction. For example, the plurality of gate lines GL may extend substantially parallel to one another in the X-axis direction.

The plurality of data lines DL may supply the data voltages received from the display driving circuit 200 to the plurality of display pixels SP. The plurality of data lines DL may extend in the Y-axis direction and may be spaced apart from each other in the X-axis direction. For example, the plurality of data lines DL may extend substantially parallel to one another in the Y-axis direction.

In addition, control lines such as a. The control lines may supply pixel driving control signals such as an emission control signal or an initialization signal received from the display driving circuit 200, to the plurality of display pixels SP. The control lines may extend in the display area DA in the Y-axis direction and may be spaced apart from each other in the X-axis direction.

The plurality of power lines VL may supply a power voltage received from the display driving circuit 200 to the plurality of display pixels SP. For example, the plurality of power lines VL may supply high-potential and low-potential power voltages received from the display driving circuit 200 to the plurality of display pixels SP. Here, the power voltage may be at least one of a high-potential driving voltage, a low-potential driving voltage, an initialization voltage, or a reference voltage. The plurality of power lines VL may extend in the Y-axis direction and may be spaced apart from each other in the X-axis direction. For example, the plurality of power lines VL may extend substantially parallel to one another in the Y-axis direction.

The non-display area NDA may surround at least a portion of the display area DA. The non-display area NDA may include a gate driving circuit 210, fan-out lines FOL, and gate control lines GCL. The gate driving circuit 210 may generate a plurality of gate signals based on the gate control signal, and may sequentially supply the plurality of gate signals to the plurality of gate lines GL according to a set order.

The fan-out lines FOL may extend from the display driving circuit 200 to the display area DA. The fan-out lines FOL may supply the data voltage received from the display driving circuit 200 to the plurality of data lines DL.

The gate control line GCL may extend from the display driving circuit 200 to the gate driving circuit 210. The gate control line GCL may supply the gate control signal received from the display driving circuit 200 to the gate driving circuit 210.

The display driving circuit 200 may supply a data voltage to the data line DL through the fan-out lines FOL. The data voltage may be supplied to the plurality of display pixels SP and may determine the luminance of the plurality of display pixels SP. The display driving circuit 200 may supply the gate control signal to the gate driving circuit 210 through the gate control line GCL. Further, the display driving circuit 200 may supply pixel driving control signals to the respective control lines through separate fan-out lines connected to the control lines of the display area DA.

When the display driving circuit 200 for controlling the driving of the first and second bio-signal detection panels FAS1 and FAS2 is disposed separately on the circuit board 300, the display driving circuit 200 may supply the data voltage to the data lines DL of the first and second bio-signal detection panels FAS1 and FAS2 through the fan-out lines FOL. Additionally, the gate control signal may be supplied to the gate driving circuits of the first and second bio-signal detection panels FAS1 and FAS2 through the gate control line GCL.

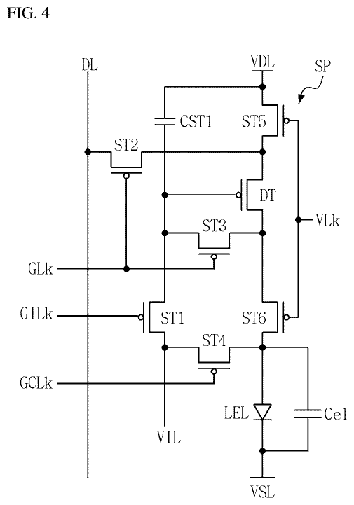

FIG. 4 is a circuit diagram of an embodiment illustrating display pixels formed in the display areas of the main display panel and the sub-display panel.

Referring to FIG. 4, the display pixels SP respectively disposed in the first and second emission areas FS1 and FS3 of the first and second bio-signal detection panels FAS1 and FAS2 may be connected to a plurality of gate lines GL, that is, a k^{th} display initialization line GILk, a k^{th} display scan line GLk, a k^{th} display control line GCLk, and a k^{th} emission control line VLk. Each of the gate lines GL may include the k^{th} display initialization line GILk, the k^{th} display scan line GLk, the k^{th} display control line GCLk, and the k^{th} emission control line VLk. Here, k is a positive integer.

Each of the display pixels SP may be divided into a light emitting portion and a pixel driver, where the light emitting portion includes a light emitting element LEL.

The pixel driver may include a driving transistor DT, switch elements, and a capacitor CST1, where the switch elements may include first to sixth transistors ST1, ST2, ST3, ST4, ST5, and ST6.

The driving transistor DT may include a gate electrode, a first electrode, and a second electrode. The driving transistor DT may control a drain-source current Ids (hereinafter, referred to as “driving current”) flowing between the first electrode and the second electrode according to a data voltage applied to the gate electrode. The driving current Ids flowing through a channel of the driving transistor DT may be proportional to the square of the difference between a threshold voltage and a voltage Vgs between the first electrode and the gate electrode of the driving transistor DT, as shown in Eq. (1):

$$Ids = {k}^{′} \times {( Vsg - Vth )}^{2} \qquad ( 1 )$$

In Eq. (1), k′ is a proportional coefficient determined by the structure and physical characteristics of the driving transistor, Vsg is a voltage between the first electrode and the gate electrode of the driving transistor, and Vth is a threshold voltage of the driving transistor.

The light emitting element LEL may emit light upon receiving the driving current Ids. As the driving current Ids increases, the amount of light emitted from the light emitting element LEL may increase.

The light emitting element LEL may be an organic light emitting diode including an organic light emitting layer disposed between an anode electrode and a cathode electrode. Alternatively, the light emitting element LEL may be an inorganic light emitting element including an inorganic semiconductor disposed between an anode electrode and a cathode electrode. Alternatively, the light emitting element LEL may be a quantum dot light emitting element including a quantum dot light emitting layer disposed between an anode electrode and a cathode electrode. Alternatively, the light emitting element LEL may be a micro light emitting element including a micro light emitting diode disposed between an anode electrode and a cathode electrode.

The anode electrode of the light emitting element LEL may be connected to a first electrode of the fourth transistor ST4 and a second electrode of the sixth transistor ST6, and the cathode electrode of the light emitting element LEL may be connected to the second driving voltage line VSL. A parasitic capacitance Cel may be formed between the anode electrode and the cathode electrode of the light emitting element LEL.

The first transistor ST1 may be turned on by the display initialization signal of the k^{th} display initialization line GILk and may connect the gate electrode of the driving transistor DT to the third driving voltage line VIL. Accordingly, the third driving voltage VINT of the third driving voltage line VIL may be applied to the gate electrode of the driving transistor DT. The gate electrode of the first transistor ST1 may be connected to the k^{th} display initialization line GILk, the first electrode thereof may be connected to the gate electrode of the driving transistor DT, and the second electrode thereof may be connected to the third driving voltage line VIL.

The second transistor ST2 may be turned on by the display scan signal of the k^{th} display scan line GLk and may connect the first electrode of the driving transistor DT to the data line DL. Accordingly, the data voltage of the data line DL may be applied to the first electrode of the driving transistor DT. The gate electrode of the second transistor ST2 may be connected to the k^{th} display scan line GLk, the first electrode thereof may be connected to the first electrode of the driving transistor DT, and the second electrode thereof may be connected to the data line DL.

The third transistor ST3 may be turned on by the display scan signal of the k^{th} display scan line GLk and may connect the gate electrode of the driving transistor DT to the second electrode thereof. When the gate electrode of the driving transistor DT is connected to the second electrode thereof, the driving transistor DT may be driven as a diode. The gate electrode of the third transistor ST3 may be connected to the k^{th} display scan line GLk, the first electrode thereof may be connected to the second electrode of the driving transistor DT, and the second electrode thereof may be connected to the gate electrode of the driving transistor DT.

The fourth transistor ST4 may be turned on by the display control signal of the k^{th} display control line GCLk and may connect the anode electrode of the light emitting element LEL to the third driving voltage line VIL. The third driving voltage of the third driving voltage line VIL may be applied to the anode electrode of the light emitting element LEL. The gate electrode of the fourth transistor ST4 is connected to the k^{th} display control line GCLk, the first electrode thereof is connected to the anode electrode of the light emitting element LEL, and the second electrode thereof is connected to the third driving voltage line VIL.

The fifth transistor ST5 may be turned on by the emission signal of the k^{th} emission control line VLk and may connect the first electrode of the driving transistor DT to the first driving voltage line VDL. The gate electrode of the fifth transistor ST5 is connected to the k^{th} emission control line VLk, the first electrode thereof is connected to the first driving voltage line VDL, and the second electrode thereof is connected to the first electrode of the driving transistor DT.

The sixth transistor ST6 is disposed between the second electrode of the driving transistor DT and the anode electrode of the light emitting element LEL. The sixth transistor ST6 may be turned on by the emission control signal of the k^{th} emission control line VLk and may connect the second electrode of the driving transistor DT to the anode electrode of the light emitting element LEL. The gate electrode of the sixth transistor ST6 is connected to the k^{th} emission control line VLk, the first electrode thereof is connected to the second electrode of the driving transistor DT, and the second electrode thereof is connected to the anode electrode of the light emitting element LEL.

When both the fifth transistor ST5 and the sixth transistor ST6 are turned on, the driving current Ids of the driving transistor DT according to the data voltage applied to the gate electrode of the driving transistor DT may flow to the light emitting element LEL.

The capacitor CST1 may be formed between the gate electrode of the driving transistor DT and the first driving voltage line VDL. The first capacitor electrode of the capacitor CST1 may be connected to the gate electrode of the driving transistor DT, and the second capacitor electrode thereof may be connected to the first driving voltage line VDL.

When the first electrode of each of the driving transistor DT and the first to sixth transistors ST1 to ST6 is a source electrode, the second electrode thereof may be a drain electrode. Alternatively, when the first electrode of each of the driving transistor DT and the first to sixth transistors ST1 to ST6 is a drain electrode, the second electrode thereof may be a source electrode.

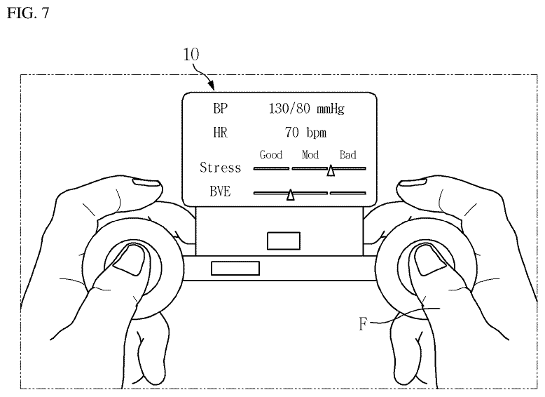

An active layer of each of the driving transistor DT and the first to sixth transistors ST1 to ST6 may be formed of any one of polysilicon, amorphous silicon, or an oxide semiconductor. In FIG. 7, the first to sixth transistors ST1 to ST6, and the driving transistor DT have been described as being formed of a P-type MOSFET, but the present disclosure is not limited thereto. For example, the first to sixth transistors ST1 to ST6, and the driving transistor DT may be formed as an N-type MOSFET. Alternatively, at least one of the first to sixth transistors ST1 to ST6 may be formed of an N-type MOSFET.

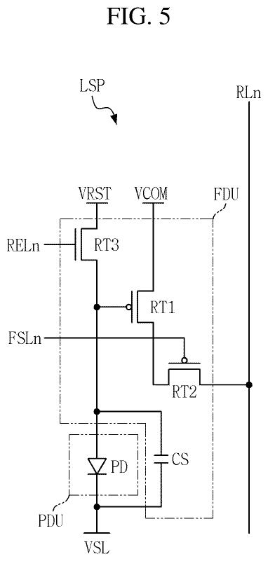

FIG. 5 is a circuit diagram of an embodiment of a light sensing pixel formed in a light sensing area of a sub-display panel.

Referring to FIG. 5, the light sensing pixels LSP may be disposed in the first and second light sensing areas FS2 and FS4 respectively formed on the first and second bio-signal detection panels FAS1 and FAS2.

Specifically, the light sensing pixels LSP may be arranged in the first and second light sensing areas FS2 and FS4 may be electrically connected to an nth sensing reset line RSLn, an nth light sensing scan line FSLn, and an nth light sensing line RLn, respectively. Here, n may be a positive integer.

The light sensing pixels LSP may sense light and generate a light sensing signal. The sensed light may be light reflected from the user's body part. For example, the sensed light may be light reflected from the user's finger, palms, or wrists. However, embodiments of the present disclosure are not limited thereto.

Each of the light sensing pixels LSP may be reset by a reset signal from the nth sensing reset line RSLn, and may transmit a light sensing signal to each nth light sensing line RLn in response to the sensing scan signal from the nth light sensing scan line FSLn.

The light sensing pixels LSP may be divided into a light sensing unit PDU and a sensing driver FDU. The light sensing unit PDU may include a light sensing element PD. The sensing driver FDU may include first to third sensing transistors RT1 to RT3 and a sensing capacitor (SC). Here, the sensing capacitor SC may be formed in parallel with the light sensing element PD.

The first sensing transistor RT1 of the sensing driver FDU may allow a light sensing current to flow according to the voltages of the light sensing element PD and the sensing capacitor SC. The amount of the light sensing current may vary depending on a voltage applied to the light sensing element PD and the sensing capacitor SC. The gate electrode of the first sensing transistor RT1 may be connected to the second electrode of the light sensing element PD. The first electrode of the first sensing transistor RT1 may be connected to a common voltage source Vcom to which a common voltage may be applied. The second electrode of the first sensing transistor RT1 may be connected to the first electrode of the second sensing transistor RT2.

When the sensing scan signal of a gate-on voltage is applied to the nth light sensing scan line FSLn, the second sensing transistor RT2 may allow the light sensing current of the first sensing transistor RT1 to flow to the nth light sensing line RLn. In this case, the nth light sensing line RLn may be charged with a sensing voltage by the light sensing current. The gate electrode of the second sensing transistor RT2 may be connected to the nth light sensing scan line FSLn, the first electrode thereof may be connected to the second electrode of the first sensing transistor RT1, and the second electrode thereof may be connected to the nth light sensing line RLn.

When a reset signal of the gate-on voltage is applied to the nth light sensing scan line FSLn, the third sensing transistor RT3 may reset the voltages of the light sensing element PD and the sensing capacitor SC to a reset voltage of a reset voltage source VRST. The gate electrode of the third sensing transistor RT3 may be connected to the sensing reset line RSL, the first electrode thereof may be connected to the reset voltage source VRST, and the second electrode thereof may be connected to the second electrode of the light sensing element PD.

Referring to FIG. 5, the first sensing transistor RT1 and the second sensing transistor RT2 may be formed of a P-type metal oxide semiconductor field effect transistor (MOSFET), and the third sensing transistor RT3 may be formed of an N-type MOSFET. However, embodiments of the present disclosure are not limited thereto, and the sensing transistors may be selectively formed in the same type or different types. Further, any one of the first electrode or the second electrode of each of the first sensing transistor RT1, the second sensing transistor RT2, and the third sensing transistor RT3 may be the source electrode and a remaining one may be the drain electrode.

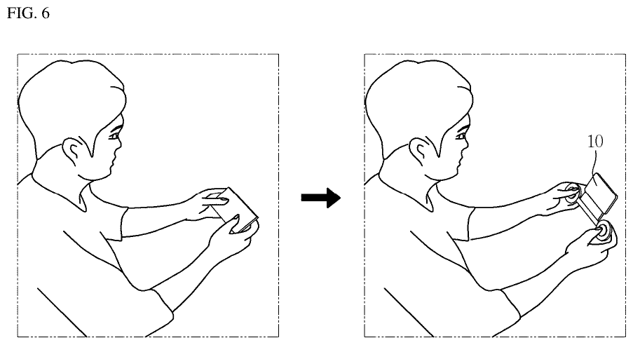

FIG. 6 is a diagram illustrating a biometric information detection process using an apparatus according to an embodiment. Further, FIG. 7 is a diagram illustrating a bio-signal detection process and biometric information confirmation process using the apparatus of FIG. 6.

Referring to FIG. 6, the display driving circuit 200 of the display device 10 may display an application program interface for pulse wave signal detection on the main display panel 100 and may cause the display pixels SP to emit light in the first and second emission areas FS1 and FS3 of the first and second bio-signal detection panels FAS1 and FAS2.

In a case that the user contacts the first and second bio-signal detection panels FAS1 and FAS2 of the display device 10 with respective body parts, such as left and right thumbs, a bio-signal such as a pulse wave signal may be detected through the first and second bio-signal detection panels FAS1 and FAS2.

The biometric information detection circuit 400 may receive optical signals, that is, light sensing signals, from the light sensing pixels LSP respectively arranged in the first and second light sensing areas FS2 and FS4 through light sensing lines ERL.

The biometric information detection circuit 400 may detect the first and second pulse wave signals (PPG signals) respectively corresponding to the light sensing signals received for each of the first and second light sensing areas FS2 and FS4. The detection of the first and second pulse wave signals may be performed in real time. The biometric information detection circuit 400 may store the first and second pulse wave signals as digital signal data. Each of the pulse wave signals may be a signal corresponding to the magnitude and magnitude change of the light sensing signals. The biometric information detection circuit 400 may display the first and second pulse wave signals detected in real time in a graph-type graphic form in the display window of the application program.

The biometric information detection circuit 400 may analyze a high pulse magnitude change and a low pulse magnitude change for at least one pulse wave signal among the first and second pulse wave signals respectively detected through the first and second light sensing areas FS2 and FS4. The analysis may be performed in real time. An average magnitude value of high pulses and an average magnitude value of low pulses may be calculated. The calculation may be performed in real time. In addition, the biometric information detection circuit 400 may select a pulse wave signal among the first and second pulse wave signals as the pulse wave signal for biometric information detection, according to comparison analysis results between the average magnitude value of high pulses and a preset high threshold value, and between the average magnitude value of low pulses and a preset low threshold value. For example, among the first and second pulse wave signals, amplitude characteristics of the second pulse wave signals may be detected to be smaller than amplitude characteristics of other first pulse wave signals. In this case, the first pulse wave signal may be selected as the pulse wave signal for biometric information detection.

FIG. 8 is a diagram illustrating a biometric information detection process using an apparatus according to an embodiment.

Referring to FIG. 8, the user may bring the first and second bio-signal detection panels FAS1 and FAS2 of the display device 10 into contact with any body part such as the back of the hand, a wrist, or an arm, and may cause a bio-signal such as a pulse wave signal to be detected through the first and second bio-signal detection panels FAS1 and FAS2.

The biometric information detection circuit 400 may receive the light sensing signals from the light sensing pixels LSP respectively arranged in the first and second light sensing areas FS2 and FS4. The biometric information detection circuit 400 may detect the first and second pulse wave signals (PPG signals) respectively corresponding to the light sensing signals received from each of the first and second light sensing areas FS2 and FS4, and may store the first and second pulse wave signals as digital signal data. The biometric information detection circuit 400 may select a pulse wave signal among the first and second pulse wave signals as the pulse wave signal for biometric information detection by analyzing the amplitude change characteristics or the like for the first and second pulse wave signals.

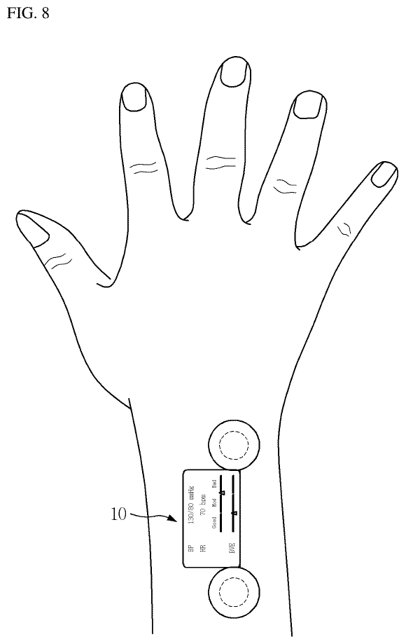

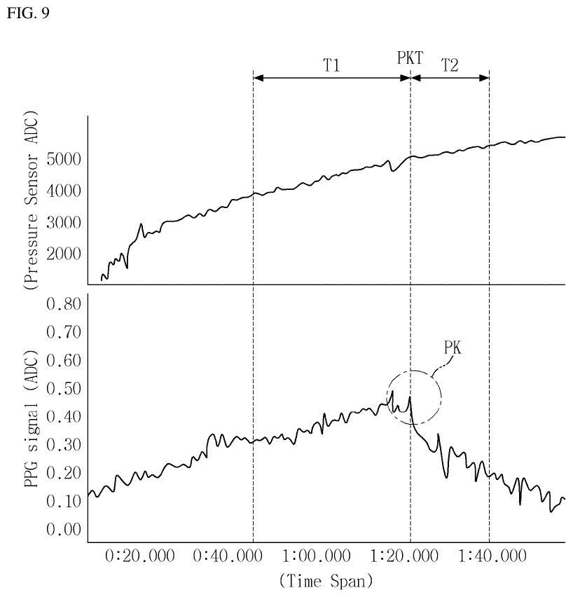

FIG. 9 is a graph illustrating a method for calculating blood pressure information among biometric information according to an embodiment.

Referring now to a typical cardiac cycle, during systole, blood is ejected from the left ventricle of the heart and moves to the peripheral tissues, and the blood volume in the arterial side may increase. Further, when the heart contracts, red blood cells carry oxygen bound to hemoglobin to peripheral tissues throughout the body. When the heart relaxes, the heart receives a partial influx of blood from the peripheral tissues.

When light is irradiated to peripheral blood vessels, the irradiated light may be absorbed by the peripheral tissues. Light absorbance may depend on hematocrit and blood volume. The light absorbance may have a maximum value when the heart contracts and may have a minimum value when the heart relaxes. Light sensed by the light sensing element PD may be low when the heart contracts and may be high when the heart relaxes.

FIG. 9 illustrates an example of measuring blood pressure while sensing a contact force of a touch applied by the user to the first and second light sensing areas. When the user brings fingers (see for example, finger F, FIG. 7) into contact with the first and second light sensing areas FS2 and FS4 in a blood pressure measurement mode, the pressure applied by the user to the first and second light sensing areas may gradually increase, reach a high value, and then gradually decrease. When the contact force of the touch increases, blood vessels may be narrowed, resulting in reduced or no blood flow. When the contact force decreases, the blood vessels may expand, and blood flow may increase. A further decrease of the contact force may result in greater blood flow. Therefore, the change in the amount of light sensed by at least one light sensing pixel LSP may be proportional to the change in blood flow. Accordingly, the biometric information detection circuit 400 may generate pulse wave signals PPG according to the pressure applied by a user based on a pressure data value (ADC of the pressure sensing unit) and the optical signal (PPG signal ratio). The pressure data value may be calculated by a pressure sensing unit and converted into a digital signal. The optical signal (PPG signal ratio) may be determined according to the amount of light sensed by the light sensing element PD. The pulse wave signals may have a waveform oscillating according to the cardiac cycle.

The biometric information detection circuit 400 may estimate blood pressure of the blood vessels of the user based on time differences between time points PKT corresponding to peaks PK of the calculated pulse wave signals and time points corresponding to peaks of the filtered pulse wave. For example, the biometric information detection circuit 400 may estimate blood pressure of the blood vessels of the user's finger F. The biometric information detection circuit 400 may calculate pulse wave signals during preset periods T1 and T2 before and after the time points PKT corresponding to the peaks PK of the calculated pulse wave signal, and may detect blood pressure according to differences between the pulse wave signals. Among the estimated blood pressure values, a maximum blood pressure value may be determined as a systolic blood pressure value, and a minimum blood pressure value may be determined as a diastolic blood pressure value. Further, additional blood pressure values, such as an average blood pressure value, be calculated using the estimated blood pressure values.

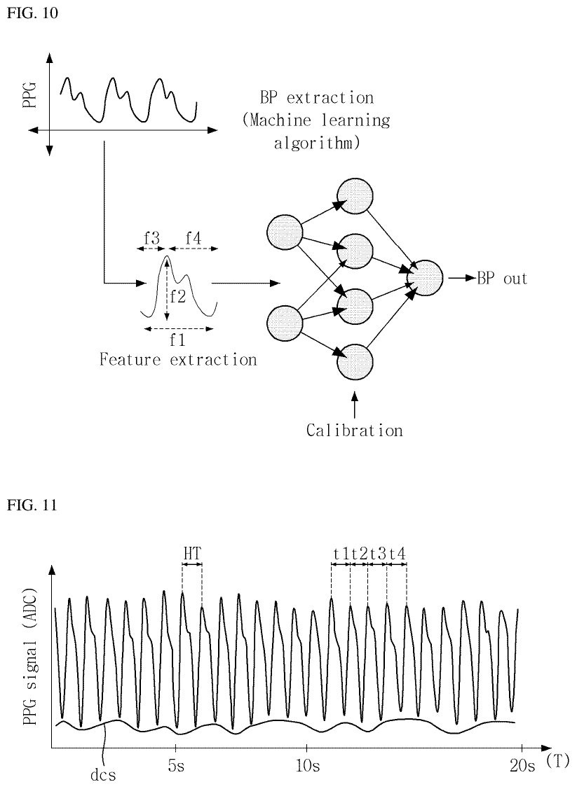

FIG. 10 is a graph illustrating a method for calculating blood pressure information among biometric information according to an embodiment.

Referring to FIG. 10, the biometric information detection circuit 400 may detect and analyze the features of the pulse wave signals without sensing the user's contact force, and may predict the user's blood pressure information by using a machine learning algorithm using a preset calibration value.

The biometric information detection circuit 400 may compare characteristic change information of the pulse wave signals with characteristic information of reference calibration values, and may generate a comparison result. The characteristic change information may be extracted as features of the pulse wave signals. The comparison result may be converted to a database as learning data. The biometric information detection circuit 400 may generate the comparison result by comparing characteristic information such as a pulse width f1 (e.g., a contraction and relaxation cycle) and frequency information, a systolic amplitude f2 (e.g., a systolic blood pressure value), a systolic pulse width f3 (e.g., a systolic cycle) change information, and a diastolic pulse width f4 (e.g., a diastolic cycle) change information of the reference calibration values, with feature changes according to the pulse wave signals, that is, the pulse width f1 (e.g., a contraction and relaxation cycle) and frequency information, the systolic amplitude f2 (e.g., a systolic blood pressure value), the systolic pulse width f3 (e.g., a systolic cycle) change information, and the diastolic pulse width f4 (e.g., a diastolic cycle) change information of the user. The comparison result may be converted to a database as learning data.

The biometric information detection circuit 400 may use a machine learning algorithm to predict the biometric information. For example, the biometric information detection circuit 400 may use a machine learning algorithm to predict a blood pressure, a heart rate, heart rate variability, a respiratory rate, blood vessel elasticity, a cardiovascular disease analysis result, or oxygen saturation according to the change rate or change magnitude in the characteristic information of the pulse wave signals, in comparison with the characteristic information of the reference calibration values. In addition, the biometric information detection circuit 400 may use a machine learning algorithm to analyze each of a high pulse period and a high pulse period change, a high pulse magnitude and a magnitude change, a low pulse magnitude and a magnitude change, the waveform change of the high pulse, the peak arrival period of the high pulse, and the pulse magnitude difference between the pulse wave signals respectively detected using green light and red light of the pulse wave signals, in comparison with the characteristic information of the reference calibration values. Further, according to the result of each analysis, biometric information including, for example, a blood pressure, a heart rate, heart rate variability, a respiratory rate, blood vessel elasticity, a cardiovascular disease analysis result, or oxygen saturation may be obtained.

The biometric information detection circuit 400 may estimate blood pressure of the blood vessels of the finger based on time differences between time points corresponding to peaks of the pulse wave signals and time points corresponding to peaks of the filtered pulse wave. For example, pulse wave signals for a preset period before and after the time points corresponding to the peaks of the pulse wave signals may be calculated, and blood pressures according to the differences in the pulse wave signals may be predicted. Among the estimated blood pressure values, a maximum blood pressure value may be determined as a systolic blood pressure value, and a minimum blood pressure value may be determined as a diastolic blood pressure value. Further, additional blood pressure values such as an average blood pressure value or the like may be calculated using the estimated blood pressure values.

FIG. 11 is a graph illustrating a method for calculating information on a heart rate and respiration among biometric information according to an embodiment.

Referring to FIG. 11, the biometric information detection circuit 400 may sample pulse wave signals during a preset sampling period before and after the time points PKT corresponding to the peaks PK of the pulse wave signal, and may detect a high pulse generation cycle HT of the sampled pulse wave signals PPG. Further, the number of high pulse generation for a preset reference period (for example, 60 seconds) may be counted for the sampled pulse wave signals PPG to detect biometric information on the heart rate cycle and the heart rate HR.

The biometric information detection circuit 400 may detect a generation cycle HT and a number of heart rate cycle t changes of high pulses for a preset reference period for the peaks PK of the pulse wave signal. For example, the biometric information detection circuit 400 may detect the generation cycle HT and the heart rate cycle changes t1 to t4 of high pulses for each preset reference period for the peaks PK of the pulse wave signal to detect heart rate variability HRV according to the heart rate cycle change rate.

The biometric information detection circuit 400 may sequentially detect the generation cycle and the magnitude value of low pulses of the sampled pulse wave signals PPG. The change cycle of a magnitude value des of low pulses may be detected every preset reference period (for example, 60 seconds) to detect the respiratory change state and a respiratory rate RR of the user. At this time, the cycle in which the magnitude value des of low pulses increases and the cycle in which the magnitude value des of low pulses decreases may be analyzed to detect the respiratory change state and the respiratory rate RR of the user using the increasing cycle and the decreasing cycle of the magnitude value des of low pulses.

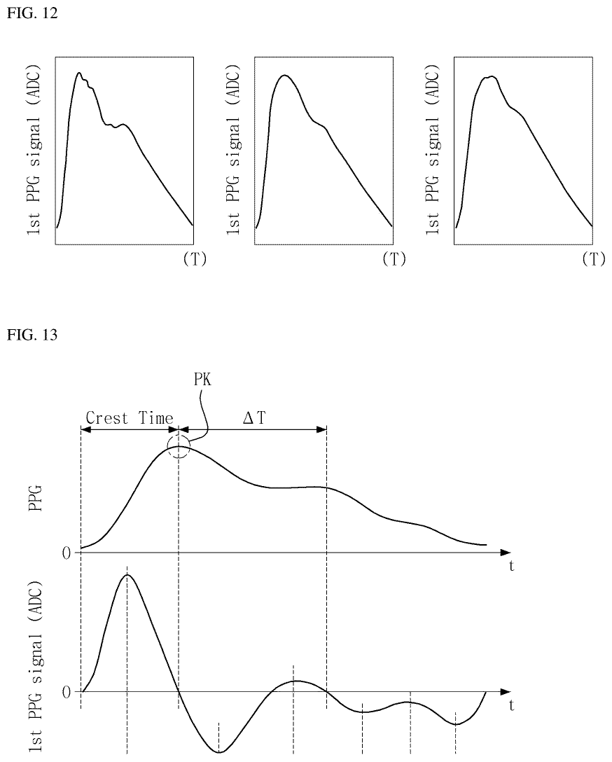

FIG. 12 is a graph illustrating a method for calculating information on blood vessel elasticity among biometric information according to an embodiment.

Referring to FIG. 12, the biometric information detection circuit 400 may set and obtain blood vessel elasticity BVE by expanding and analyzing the high pulse variation of the sampled pulse wave signals.

When the blood flow increases due to heartbeat, the pulse wave signal may be changed to a high pulse form, and when the blood flow decreases, the pulse wave signal may be changed to a low pulse form. If the blood flow changes rapidly due to the shape of the blood vessel during the period in which the blood flow increases or decreases, the change in the blood flow may be quickly relaxed or slowed depending on the elasticity of the blood vessel. Accordingly, the biometric information detection circuit 400 may set and obtain the blood vessel elasticity BVE using a value corresponding to the magnitude of changes in high pulses by expanding and analyzing the high pulse change form of the pulse wave signals.

FIG. 13 is a graph illustrating a method for calculating information on a cardiovascular disease among biometric information according to an embodiment.

Referring to FIG. 13, the biometric information detection circuit 400 may set and obtain a cardiovascular disease evaluation score (or a cardiovascular health analysis result score) by differentiating, expanding and analyzing the high pulse change form of the sampled pulse wave signals PPG. For example, the biometric information detection circuit 400 may detect a period (Crest Time) in which the pulse wave signals PPG reach the peak PK in a high pulse form, and time variation ΔT in which the pulse wave signals PPG fall compared to the period (Crest Time) in which the pulse wave signals PPG reach the peak PK. As the period (Crest Time) in which the pulse wave signals PPG reach the peak PK in a high pulse form increases, the risk of heart disease may increase. Accordingly, the biometric information detection circuit 400 may set and obtain the cardiovascular disease evaluation score (or the cardiovascular health analysis result score) in inverse proportion to the period (Crest Time) in which the pulse wave signals PPG reach the peak PK in a high pulse form.

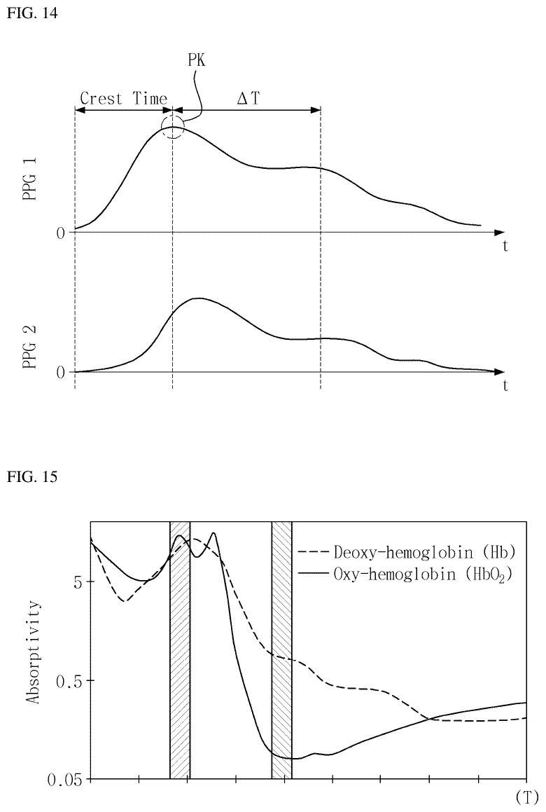

FIG. 14 is a graph illustrating a method for calculating information on a cardiovascular disease among biometric information according to an embodiment.

Referring to FIG. 14, the biometric information detection circuit 400 may detect pulse wave signals PPG1 of a left-hand finger and pulse wave signals PPG2 of a right-hand finger, and may set and obtain the cardiovascular disease evaluation score (or the cardiovascular health analysis result score) by differentiating and expanding the high pulse fluctuation form for each of the pulse wave signals PPG1 and PPG2 detected from the fingers. Further, based on a preset analysis algorithm, the evaluation score (or the cardiovascular health analysis result score) detected from the pulse wave signals PPG2 of a finger may be obtained as the final result.

For example, for each of the pulse wave signals PPG1 and PPG2 detected from two different fingers, respectively, a period (Crest Time) during which each of the pulse wave signals PPG1 and PPG2 reaches the peak PK in a high pulse form, and the time variation ΔT during which the pulse wave signals PPG decrease as compared with the period (Crest Time) during which the peak PK is reached, may be detected. Further, after obtaining an evaluation score (or a cardiovascular health analysis result score) for each of the pulse wave signals PPG1 and PPG2 detected from the fingers, the lower evaluation score may be selected and outputted as the final evaluation score.

FIG. 15 is a graph illustrating a method for calculating information on oxygen saturation among biometric information according to an embodiment.

Referring to FIG. 15, when the heart contracts, red blood cells may carry more hemoglobin bound oxygen to peripheral tissues. On the other hand, when the heart relaxes, the heart may receive a partial influx of blood from the peripheral tissues. Using this, the biometric information detection circuit 400 may detect a deoxy-hemoglobin (Hb) value using the magnitude change of the pulse wave signals PPG detected by the green light, and detects a HbO₂ (Oxy-hemoglobin) value using the magnitude change of the pulse wave signals PPG detected by the red light.

The biometric information detection circuit 400 may detect the oxygen saturation (SpO₂) using the following Eq. (2):

$${Sp O}_{2} = {Hp O}_{2} / {Sp O}_{2} + Hb \qquad ( 2 )$$

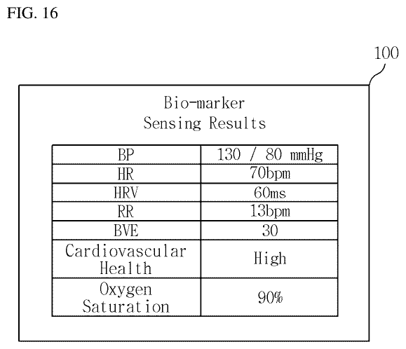

FIG. 16 is a diagram illustrating biometric information measurement results through the main display panel of the display device according to an embodiment.

Referring to FIG. 16, the biometric information detection circuit 400 may transmit the biometric information such as the blood pressure BP, the heart rate HR, the heart rate variability HRV, the respiratory rate RR, the blood vessel elasticity BVE, the cardiovascular disease (or the cardiovascular health analysis result score), or the oxygen saturation (SpO₂) to the display driving circuit 200.

The display driving circuit 200 may display the biometric information on the application program screen through the main display panel 100 of the display device 10. The biometric information may include one or more of blood pressure BP, heart rate HR, heart rate variability HRV, respiratory rate RR, blood vessel elasticity BVE, cardiovascular disease (or cardiovascular health analysis result score), or oxygen saturation (SpO₂).

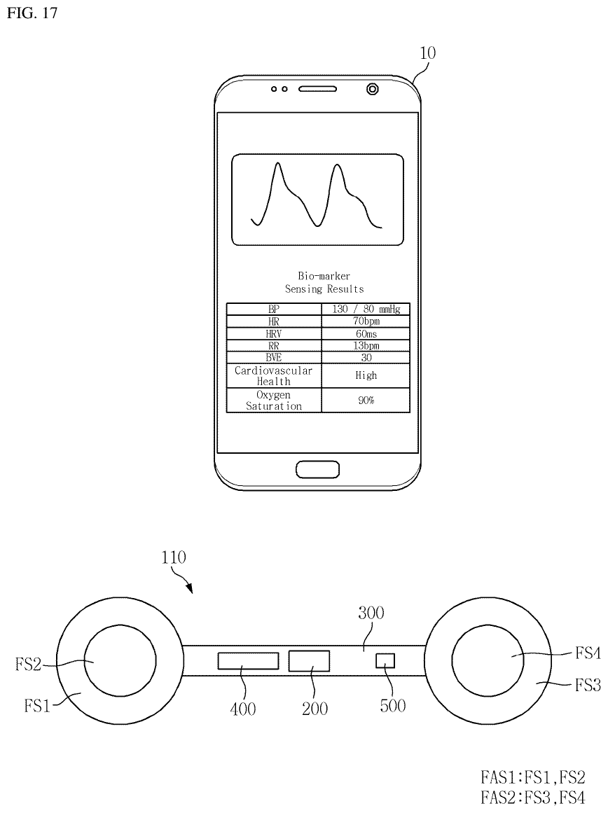

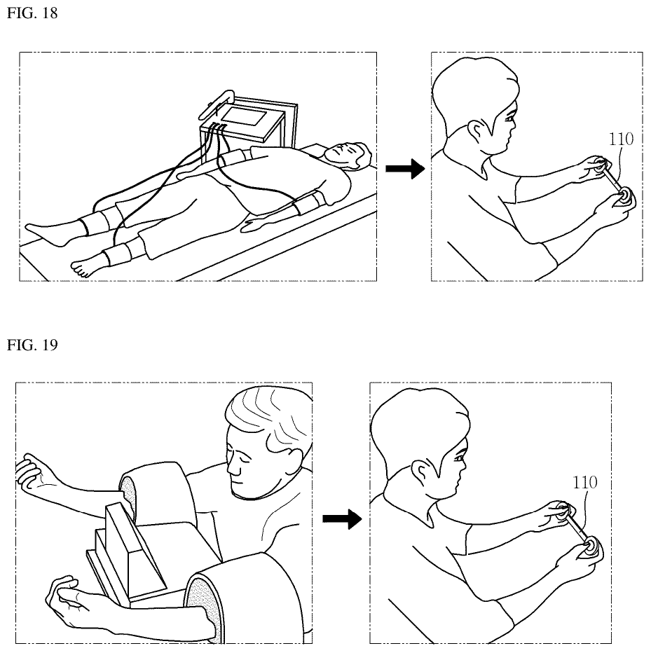

FIG. 17 is a plan view showing an apparatus according to an embodiment of the present disclosure.

Referring to FIG. 17, the sub-display panel 110 may include the first bio-signal detection panel FAS1 including the first emission area FS1 in which the display pixels SP are arranged and the first light sensing area FS2 in which the light sensing pixels LSP are arranged. The first bio-signal detection panel FAS1 may be coupled or assembled to a first end portion of the circuit board 300.

In addition, the sub-display panel 110 may further include the second bio-signal detection panel FAS2 including the second emission area FS3 in which the display pixels SP are arranged and the second light sensing area FS4 in which the light sensing pixels LSP are arranged. The second bio-signal detection panel FAS2 may be coupled or assembled to a second end portion of the circuit board 300.

The display driving circuit 200 may generate electrical signals such as gate signals, data voltages, and driving voltages for driving the first and second bio-signal detection panels FAS1 and FAS2.

The biometric information detection circuit 400 may measure the user's biometric information by using light sensing signals received through at least one bio-signal detection panel among the first and second bio-signal detection panels FAS1 and FAS2.

Each of the biometric information detection circuits 400, including the display driving circuit 200, may be formed as an integrated circuit (IC), and may be attached onto the circuit board 300 by using a chip on glass (COG) method, a chip on plastic (COP) method, or an ultrasonic bonding method.

The main display panel 100 may be disposed in a separate mobile display device such as a smartphone or tablet device, in a state separated from the first and second bio-signal detection panels FAS1 and FAS2, and the circuit board 300.

The sub-display panel 110 may further include at least one short-range wireless communication module 500 that may transmit the user's biometric information detected through the biometric information detection circuit 400 to a separate mobile display device in which the main display panel 100 is formed. The short-range wireless communication module 500 may include a wireless communication module such as Wi-Fi, Bluetooth, or ZigBee.

FIG. 18 and FIG. 19 are diagrams illustrating a biometric information detection process using the apparatus of FIG. 17.

Referring to FIG. 18 and FIG. 19, the display driving circuit 200 of the sub-display panel 110 may cause the display pixels SP of the first and second emission areas FS1 and FS3 formed on the first and second bio-signal detection panels FAS1 and FAS2 to emit light.

The user may contact the first and second bio-signal detection panels FAS1 and FAS2 of the display device 10 with body parts such as a fingers, respectively, and may cause a bio-signal such as a pulse wave signal to be detected through the first and second bio-signal detection panels FAS1 and FAS2.

The biometric information detection circuit 400 may receive optical signals, that is, light sensing signals, from the light sensing pixels LSP respectively arranged in the first and second light sensing areas FS2 and FS4 through the light sensing lines ERL. The biometric information detection circuit 400 may transmit the biometric information such as the blood pressure BP, the heart rate HR, the heart rate variability HRV, the respiratory rate RR, the blood vessel elasticity BVE, the cardiovascular disease (or the cardiovascular health analysis result score), or the oxygen saturation (SpO₂) to the short-range wireless communication module 500.

The short-range wireless communication module 500 may transmit the biometric information to a separate mobile display device in which the main display panel 100 is formed. The short-range wireless communication module 500 may transmit the biometric information such as the blood pressure BP, the heart rate HR, the heart rate variability HRV, the respiratory rate RR, the blood vessel elasticity BVE, the cardiovascular disease (or the cardiovascular health analysis result score), or the oxygen saturation (SpO₂) to a separate mobile display device in which the main display panel 100 is formed.

The display driving circuit of the mobile display device may display the biometric information such as the blood pressure BP, the heart rate HR, the heart rate variability HRV, the respiratory rate RR, the blood vessel elasticity BVE, the cardiovascular disease (or the cardiovascular health analysis result score), or the oxygen saturation (SpO₂) on the application program screen.

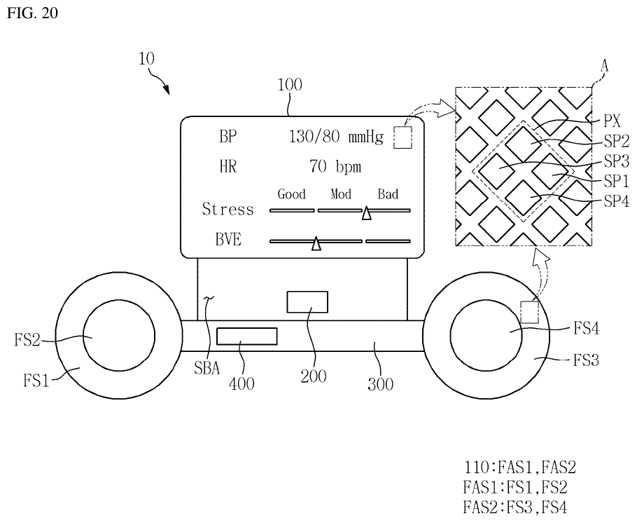

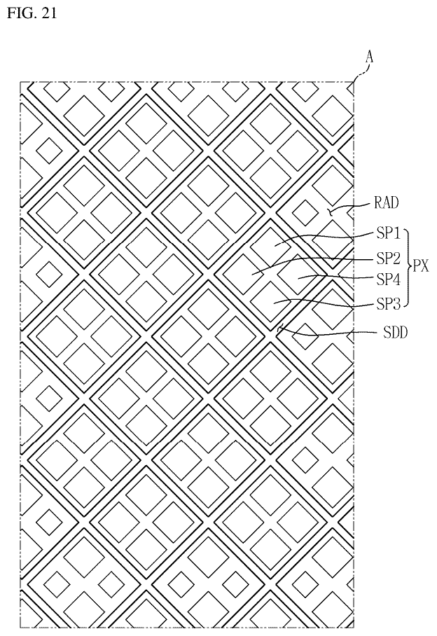

FIG. 20 is a plan view showing an apparatus according to an embodiment of the present disclosure.

Referring to FIG. 20, the sub-display panel 110 may include the first bio-signal detection panel FAS1 including the first emission area FS1 in which the display pixels SP may be arranged, and the first light sensing area FS2 in which the light sensing pixels LSP may be arranged, and the first bio-signal detection panel FAS1 may be coupled or assembled to a first end portion of the circuit board 300.

In addition, the sub-display panel 110 may further include a second bio-signal detection panel FAS2 including a second emission area FS3 in which the display pixels SP may be arranged, and a second light sensing area FS4 in which the light sensing pixels LSP may be arranged. The second bio-signal detection panel FAS2 may be coupled or assembled to a second end portion of the circuit board 300.

The first and second bio-signal detection panels FAS1 and FAS2, including the main display panel 100, may be formed flat, but are not limited thereto. For example, the first and second bio-signal detection panels FAS1 and FAS2 may include curved portions with a constant curvature or a changing curvature. In addition, the first and second bio-signal detection panels FAS1 and FAS2 may be flexible. For example, the first and second bio-signal detection panels FAS1 and FAS2 may be curved, bent, folded, stretched, or rolled.

The substrate SUB of each of the first and second bio-signal detection panels FAS1 and FAS2, including the main display panel 100, may be a base substrate or a base member. The substrate SUB may be a flexible substrate which may be bent, folded or rolled. For example, the substrate SUB may include a glass material or a metal material, but is not limited thereto. For another example, the substrate SUB may include a polymer resin such as polyimide (PI).

The display driving circuit 200 may generate electrical signals such as gate signals, data voltages, and driving voltages for driving the first and second bio-signal detection panels FAS1 and FAS2.

The biometric information detection circuit 400 may measure the user's biometric information by using light sensing signals received from at least one bio-signal detection panel among the first and second bio-signal detection panels FAS1 and FAS2.

The main display panel 100 may be electrically connected to the circuit board 300 through a flexible film.

The biometric information detection circuit 400 may receive optical signals, that is, light sensing signals, from the light sensing pixels LSP respectively arranged in the first and second light sensing areas FS2 and FS4 through light sensing lines ERL. The biometric information detection circuit 400 may detect the biometric information such as the blood pressure BP, the heart rate HR, the heart rate variability HRV, the respiratory rate RR, the blood vessel elasticity BVE, the cardiovascular disease (or the cardiovascular health analysis result score), or the oxygen saturation (SpO₂), and may transmit the biometric information to the display driving circuit 200.

The display driving circuit 200 may display the biometric information such as the blood pressure BP, the heart rate HR, the heart rate variability HRV, the respiratory rate RR, the blood vessel elasticity BVE, the cardiovascular disease (or the cardiovascular health analysis result score), or the oxygen saturation (SpO₂) on the application program screen through the main display panel 100.

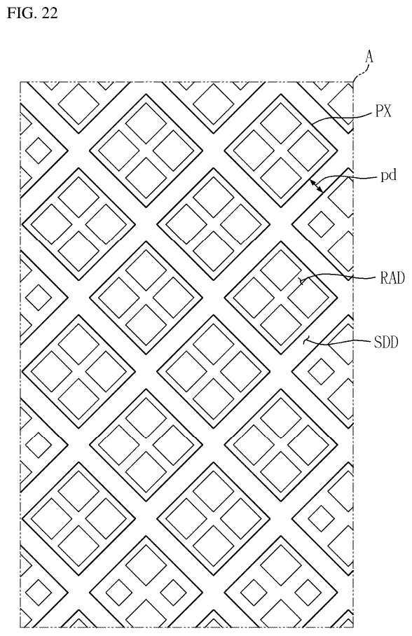

FIG. 21 is a diagram illustrating a display pixel disposition structure in area A illustrated in FIG. 20. FIG. 22 is a diagram specifically illustrating the disposition structure of display pixels according to stretch deformation of area A illustrated in FIG. 21.

FIG. 21 and FIG. 22 illustrate that the display area DA (see FIG. 2) of area A illustrated in FIG. 20 is in a contracted or stretched state due to the contracting or stretching of the main display panel 100 and the first and second bio-signal detection panels FAS1 and FAS2.

Each of the first and second bio-signal detection panels FAS1 and FAS2, including the main display panel 100, may include a curved portion having a constant curvature or a changing curvature. In addition, the first and second bio-signal detection panels FAS1 and FAS2 may be formed flexibly to be curved, bent, folded, stretched, contracted, or rolled.

The substrate SUB of each of the first and second bio-signal detection panels FAS1 and FAS2, including the main display panel 100, may be a flexible substrate which can be bent, folded, rolled, stretched or contracted.

For example, the first to fourth display pixels SP1 to SP4 may form respective groups that may be defined as respective unit pixels PX in the display area DA of the main display panel 100 or in the first and second emission areas FS1 and FS3 of the first and second bio-signal detection panels FAS1 and FAS2. Further, each of the display pixels SP1 to SP4 and each of the unit pixels PX may be arranged in a pentile matrix structure to emit light.

The first to fourth display pixels SP1 to SP4 included in each of the unit pixels PX may display an image by emitting red, green, blue, and white light, respectively. Additionally, each of the first to fourth sub-pixels SP1 to SP4 may display an image by emitting light of different colors or the same color, such as red, green, blue, or green.

The display area DA of the main display panel 100 or the first and second emission areas FS1 and FS3 of the first and second bio-signal detection panels FAS1 and FAS2 may include a plurality of rigid areas RAD and a stretchable area SDD disposed between the plurality of rigid areas RAD.

The plurality of rigid areas RAD may include the respective unit pixels PX partitioned by a partition wall. Further, each of the unit pixels PX may include the first to fourth display pixels SP1 to SP4 disposed in a pentile matrix structure. Specifically, in each of the rigid areas RAD, the first to fourth sub-pixels SP1 to SP4 constituting one unit pixel PX may be disposed in a pentile matrix structure, so that the disposition area of each unit pixel PX may be defined as each rigid area RAD.

The stretchable area SDD may be an area between the rigid areas RAD, and the stretchable area SDD may include elastic connection members formed such that a gap pd between the rigid areas RAD may be narrowed or widened. Accordingly, the area in which the elastic connection members are formed may be defined as the stretchable area SDD, and the gap pd between the respective rigid areas RAD may increase or decrease due to the stretching and contraction deformation of the elastic connection members.

Each of the unit pixels PX disposed in each of the rigid areas RAD may be electrically connected to each of the other adjacent unit pixels PX by a stretching line included in the elastic connection members. Accordingly, the gap between the unit pixels PX may also increase or decrease due to the stretching and contracting deformation of the elastic connection members including the stretching line.

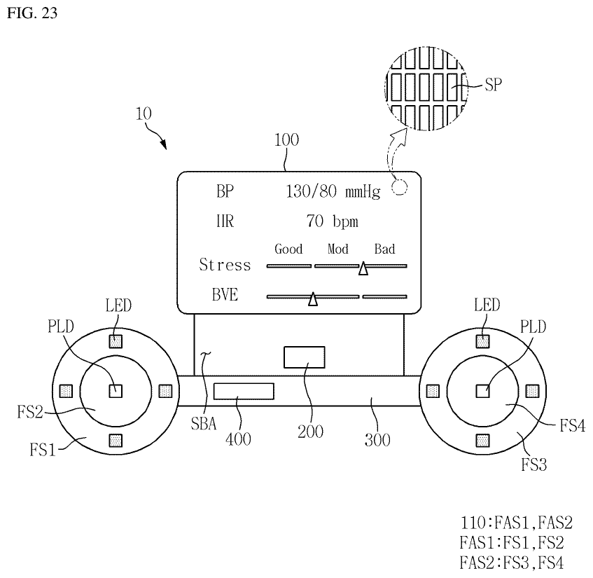

FIG. 23 is a plan view showing a display device according to an embodiment of the present disclosure.

Referring to FIG. 23, the sub-display panel 110 may include the first bio-signal detection panel FAS1 including the first emission area FS1 in which the display pixels SP may be arranged, and the first light sensing area FS2 in which the light sensing pixels LSP may be arranged. The first bio-signal detection panel FAS1 may be coupled or assembled to a first end portion of the circuit board 300.

In addition, the sub-display panel 110 may further include a second bio-signal detection panel FAS2 including a second emission area FS3 in which the display pixels SP may be arranged and a second light sensing area FS4 in which the light sensing pixels may be are arranged. The second bio-signal detection panel FAS2 may be coupled or assembled to a second end portion of the circuit board 300.

The display pixels SP respectively disposed in the first and second emission areas FS1 and FS3 of the first and second bio-signal detection panels FAS1 and FAS2 may include a pixel driver including the driving transistor DT, switch elements, and the capacitor CST1, and a light emitting portion including a light emitting diode such as a micro light emitting diode. That is, the display pixels SP may be respectively disposed in the first and second emission areas FS1 and FS3 of the first and second bio-signal detection panels FAS1 and FAS2 may emit light using a light emitting diode such as a micro light emitting diode.

The light sensing pixels LSP that sense light reflected from the user's body part to generate and output a light sensing signal may be disposed in the first and second light sensing areas FS2 and FS4 respectively formed on the first and second bio-signal detection panels FAS1 and FAS2.

Each of the light sensing pixels LSP may be configured to include the sensing driver FDU, including the first to third sensing transistors RT1 to RT3, and a sensing capacitor SC, and the light sensing unit PDU may include a photodiode PD.

The photodiode PD formed in each of the light sensing units PDU may transmit light sensing signals to the biometric information detection circuit 400 through the light sensing lines ERL.

In concluding the detailed description, those skilled in the art will appreciate that many variations and modifications can be made to embodiments without substantially departing from the principles of the present invention. Therefore, embodiments of the invention are used in a generic and descriptive sense only and not for purposes of limitation.

What is claimed is:

1. An apparatus for detecting biometric information comprising:
a sub-display panel configured to drive a plurality of display pixels to emit light, and generate light sensing signals by sensing light using a plurality of light sensing pixels;
a biometric information detection circuit configured to measure and detect biometric information using the light sensing signals received from the sub-display panel;
a main display panel configured to display at least a portion of the biometric information; and
a display driving circuit configured to control a driving timing of the plurality of light sensing pixels and the plurality of display pixels of the sub-display panel.

2. The apparatus of claim 1, wherein the sub-display panel comprises:
a first bio-signal detection panel comprising a first emission area in which first display pixels of the plurality of display pixels are arranged and a first light sensing area in which first light sensing pixels of the plurality of light sensing pixels are arranged; and
a circuit board coupled and electrically connected to a first end portion of the first bio-signal detection panel,
wherein the biometric information detection circuit and the display driving circuit are mounted on the circuit board.

3. The apparatus of claim 2, wherein the first display pixels are arranged in a matrix structure in the first emission area of the first bio-signal detection panel,
the first light sensing area of the first bio-signal detection panel is formed at a center portion of the first emission area to have a perimeter smaller than that of the first emission area, and the first light sensing pixels are arranged in a matrix structure in the first light sensing area of the first bio-signal detection panel.

4. The apparatus of claim 2, wherein the sub-display panel further comprises a second bio-signal detection panel comprising a second emission area in which second display pixels of the plurality of display pixels are arranged and a second light sensing area in which second light sensing pixels of the plurality of light sensing pixels are arranged, and
the second bio-signal detection panel is coupled and electrically connected to a second end portion of the circuit board.

5. The apparatus of claim 4, wherein the display driving circuit generates electrical signals comprising gate signals, a data voltage, and a driving voltage for driving at least one bio-signal detection panel among the first bio-signal detection panel and the second bio-signal detection panel, and
the biometric information detection circuit measures and detects the biometric information using light sensing signals received through the at least one bio-signal detection panel.

6. The apparatus of claim 4, wherein each of the first bio-signal detection panel and the second bio-signal detection panel is formed having a rounded shape or a polygonal shape in plan view.

7. The apparatus of claim 4, wherein the second display pixels are arranged in a matrix structure in the second emission area of the second bio-signal detection panel,
the second light sensing area of the second bio-signal detection panel is formed at a center portion of the second emission area to have a perimeter smaller than that of the second emission area, and the second light sensing pixels are arranged in a matrix structure in the second light sensing area of the second bio-signal detection panel.

8. The apparatus of claim 4, wherein the sub-display panel further comprises a flexible film connected to the circuit board, and
the main display panel is electrically connected to the display driving circuit through the flexible film and the circuit board.

9. The apparatus of claim 8, wherein a substrate of each of the first bio-signal detection panel and the second bio-signal detection panel is formed of a flexible substrate, and each of the first bio-signal detection panel and the second bio-signal detection panel is formed to be flexible.

10. The apparatus of claim 8, wherein the first display pixels and the second display pixels respectively disposed in the first emission area and the second emission area each comprise:
a light emitting portion comprising a light emitting diode; and
a pixel driver comprising a driving transistor, switch elements, and a capacitor and controlling a light emission operation of the light emitting diode.

11. The apparatus of claim 8, wherein each of the first light sensing pixels and the second light sensing pixels respectively disposed in the first light sensing area and the second light sensing area each comprise:
a light sensing unit comprising a photodiode; and
a sensing driver comprising first to third sensing transistors and a sensing capacitor to control a light sensing operation of the photodiode.

12. An apparatus for detecting biometric information comprising:
a sub-display panel configured to drive display pixels to emit light, and detect light sensing signals by sensing light using light sensing pixels, the sub-display panel comprising:
a biometric information detection circuit configured to measure and detect biometric information of a user using the light sensing signals received through the sub-display panel; and
a display driving circuit configured to control a driving timing of the light sensing pixels and the display pixels of the sub-display panel; and
a main display panel configured to display the biometric information,
wherein the main display panel is formed in a mobile display device in a state separated from the sub-display panel, and
the sub-display panel further comprises at least one short-range wireless communication module configured to transmit the biometric information detected through the biometric information detection circuit to the mobile display device in which the main display panel is formed.

13. An apparatus for detecting biometric information comprising:
a sub-display panel configured to drive a plurality of display pixels to emit light, and generate light sensing signals by sensing light reflected from a body part of a user using a plurality of light sensing pixels;
a biometric information detection circuit configured to measure and detect biometric information of the user using the light sensing signals received from the sub-display panel;
a main display panel configured to display at least a portion of the biometric information; and
a display driving circuit configured to control a driving timing of the plurality of light sensing pixels and the plurality of display pixels of the sub-display panel,
wherein the display driving circuit generates electrical signals comprising gate signals, a data voltage, and a driving voltage for driving the sub-display panel, and
the biometric information detection circuit measures and detects the biometric information using light sensing signals received from the sub-display panel.

14. The apparatus of claim 13, wherein the sub-display panel comprises:
a first bio-signal detection panel comprising a first emission area in which first display pixels of the plurality of display pixels are arranged and a first light sensing area in which first light sensing pixels of the plurality of light sensing pixels are arranged; and
a circuit board coupled and electrically connected to a first end portion of the first bio-signal detection panel,
wherein the biometric information detection circuit and the display driving circuit are mounted on the circuit board.

15. The apparatus of claim 14, wherein the first emission area of the first bio-signal detection panel is formed in a rounded shape or a polygonal shape in plan view, the first display pixels are arranged in a matrix structure in the first emission area of the first bio-signal detection panel,
the first light sensing area of the first bio-signal detection panel is formed at a center portion of the first emission area to have a perimeter smaller than that of the first emission area, and the first light sensing pixels are arranged in a matrix structure in the first light sensing area of the first bio-signal detection panel.

16. The apparatus of claim 14, wherein the sub-display panel further comprises a second bio-signal detection panel comprising a second emission area in which second display pixels of the plurality of display pixels are arranged and a second light sensing area in which second light sensing pixels of the plurality of light sensing pixels are arranged, and
the second bio-signal detection panel is coupled and electrically connected to a second end portion of the circuit board.

17. The apparatus of claim 16, wherein each of the first bio-signal detection panel and the second bio-signal detection panel is formed in one of a circular shape, an elliptical shape, a triangular shape, a quadrilateral shape, a pentagonal shape, or a hexagonal shape.

18. The apparatus of claim 16, wherein the second display pixels are arranged in a matrix structure in the second emission area of the second bio-signal detection panel,
the second light sensing area of the second bio-signal detection panel is formed at a center portion of the second emission area to have a perimeter smaller than that of the second emission area, and the second light sensing pixels are arranged in a matrix structure in the second light sensing area of the second bio-signal detection panel.

19. The apparatus of claim 14, wherein the sub-display panel further comprises a flexible film connected to the circuit board, and
the main display panel is electrically connected to the display driving circuit through the flexible film and the circuit board.

20. The apparatus of claim 14, wherein the main display panel is formed in a mobile display device in a state separated from the first bio-signal detection panel and the circuit board, and
the sub-display panel further comprises at least one short-range wireless communication module configured to transmit the biometric information of the user detected through the biometric information detection circuit to the mobile display device in which the main display panel is formed.
